(19) United States
(12) Patent Application Publication
(10) Pub. No.: US 2018/0228833 A1
(43) Pub. Date: Aug. 16, 2018

(54) OVINE ENOXAPARIN SODIUM, PREPARATION METHOD THEREFOR, AND APPLICATION THEREOF

(71) Applicant: SUZHOU RONNSI PHARMA CO., LTD., Suzhou (CN)

(72) Inventors: Yongsheng JIN, Suzhou (CN); Caijuan JIN, Suzhou (CN); Ningxia WANG, Suzhou (CN); Yiming YAO, Suzhou (CN)

(73) Assignee: SUZHOU RONNSI PHARMA CO., LTD., Suzhou (CN)

(21) Appl. No.: 15/752,575

(22) PCT Filed: Aug. 19, 2016
(86) PCT No.: PCT/CN2016/096016

§ 371 Date: Feb. 13, 2018

(30) Foreign Application Priority Data
Aug. 21, 2015 (CN) 201510519349.0

(51) Int. Cl. A61K 31/727; A61K 9/00

(52) U.S. Cl. CPC A61K 31/727; A61K 9/0019

(57) ABSTRACT

The present invention discloses an ovine enoxaparin sodium, a preparation method therefor, and an application thereof. The ovine enoxaparin sodium is prepared from ovine heparin. Compared with enoxaparin sodium derived from porcine intestinal mucosa heparin, both the chemical structures (disaccharide compositions) and the physiochemical properties are different. Through process of optimization and precipitation, the ovine enoxaparin sodium prepared meets the enoxaparin sodium specifications in the USP 37 and the EP 8.0 as well as specifications in current editions of the USP 39 and the EP 8.6. Also provided are two types of ovine enoxaparin sodium injections, preparation methods therefor, and anticoagulation properties in animal model. The raw material used in the preparation is ovine, which is a Halal material compared to porcine enoxaparin sodium. Therefore, the ovine enoxaparin sodium and the injections thereof are an Halal anticoagulant medicine.

### CROSS REFERENCE TO RELATED APPLICATIONS

This application is the national phase entry of International Application No. PCT/CN2016/096016, filed on Aug. 19, 2016, which is based upon and claims priority to Chinese Patent Application No. CN201510519349.0, filed on Aug. 21, 2015, the entire contents of which are incorporated herein by reference.

### TECHNICAL FIELD

The present invention relates to the field of pharmaceutical product and halal medicine development, and more particularly to an ovine-derived low molecular weight heparin-ovine enoxaparin sodium, and a preparation method therefor, and an application thereof.

### BACKGROUND OF THE INVENTION

Enoxaparin sodium (ES) is a low molecular weight heparin sodium salt, obtained by depolymerization of unfractionated heparin, and is being widely used clinically today as an anticoagulant. Currently, the USP 39 and the EP 8.6 defines that the source of enoxaparin sodium is porcine intestinal mucosa heparin; however, porcine heparin can not be halal medicine.

Halal is a special requirement for Muslim population. There is an explicit requirement on foods and medicines in Muslim theology, and people are only permitted to consume products from ruminants such as cattle, ovine and goats among mammals and prohibited from consuming products from non-ruminants such as pigs and dogs. Global Muslim population has exceeded 1.6 billions in 2013, accounting for 23% of global total population of 6.9 billions. In some countries where Muslims are dominant, such as Indonesia, Pakistan and Iran, there is a lack of Halal anticoagulant medicines in the market that meet the requirement of Muslim theology. Thus, it is particularly advantageous to develop halal low molecular weight heparin that meets the requirement of Muslim theology.

The present inventors have described in detail a method for preparing ovine enoxaparin sodium in a previous patent application (application publication No. CN 105131153 A). This ovine enoxaparin sodium disclosed by the present inventors is never been disclosed by others and is different from porcine enoxaparin sodium. The present invention will focus on disclosing ovine enoxaparin and its injections' preparation and their specific physiochemical properties and biological properties.

### SUMMARY OF THE INVENTION

The objectives of the present invention are to provide ovine enoxaparin sodium, a preparation method therefor, and an application thereof.

The objectives of the present invention are achieved by the following technical solutions.

Ovine enoxaparin sodium is prepared from ovine heparin.

Disaccharide composition of the ovine enoxaparin sodium is analyzed by SAX-HPLC after enzymatic hydrolysis by heparinases. The main disaccharide ΔUA2S-GlcNS6S (ΔIS) in the heparinases digested product is between 60%-74% from SAX-HPC spectrum, followed by two other major disaccharides ΔUA-GlcNS6S (ΔIIS) and ΔUA2S-GlcNS (ΔIIIS) with 8%-11% and 4%-7% respectively. The 3-sulfated tetradisaccharide (part of the core pentasaccharide crucial to anti-Xa and anti-IIa activities), ΔIIA-IISglu, is 1.2%-2.1%. In contrast, in porcine intestinal mucosa enoxaparin sodium, the contents of ΔIS, ΔIIS and ΔIIIS are 58%-66%, 9.5%-11.5% and 5.8%-7.8% respectively, and the content of ΔIIA-IISglu is 2.1%-2.5%.

The ovine enoxaparin sodium can be ovine-derived enoxaparin sodium and goat-derived enoxaparin sodium. In the ovine-derived enoxaparin sodium, the content of the disaccharide ΔUA2S-GlcNS6S (ΔIS) is 66%-74%, the content of the disaccharide ΔUA-GlcNS6S (ΔIIS) is 8%-10%, and the content of the disaccharide ΔUA2S-GlcNS (ΔIIIS) is 4%-6%; and in the goat-derived enoxaparin sodium, the content of the disaccharide ΔUA2S-GlcNS6S (ΔIS) is 60%-68%, the content of the disaccharide ΔUA-GlcNS6S (ΔIIS) is 9%-11%, and the content of the disaccharide ΔUA2S-GlcNS (ΔIIIS) is 5%-7%. Further, in the ovine-derived enoxaparin sodium, the content of the disaccharide ΔUA2S-GlcNS6S (ΔIS) is 66.26%, the content of the disaccharide ΔUA-GlcNS6S (ΔIIS) is 9.15%, and the content of the disaccharide ΔUA2S-GlcNS (ΔIIIS) is 6.44%; and in the goat-derived enoxaparin sodium, the content of the disaccharide ΔUA2S-GlcNS6S (ΔIS) is 63.58%, the content of the disaccharide ΔUA-GlcNS6S (ΔIIS) is 10.71%, and the content of the disaccharide ΔUA2S-GlcNS (ΔIIIS) is 10.27%.

Chemical structure of the ovine enoxaparin sodium is determined by ¹H-NMR spectrum and ¹³C-NMR spectrum. The spectra of ovine enoxaparin sodium are similar to the spectra of porcine enoxaparin sodium, but the integral of methyl peak of N-acetyl at δ2.04 ppm in ¹H-NMR spectrum and δ24.9 ppm in ¹³C-NMR spectrum of ovine enoxaparin sodium is smaller than the integral of corresponding methyl peak in porcine enoxaparin sodium, indicating that less N-acetyl group is present in the former. For the NMR method, advanced 2D-NMR analysis such as HSQC-NMR is more preferred, so that differences in some specific sugar chain structures can be explicitly determined.

The sulfate to carboxylate ratio in the ovine enoxaparin sodium is determined using the method from the USP 37. The sulfate to carboxylate ratio reflects sulfate modification on the sugar chain. The sulfate to carboxylate ratio in ovine enoxaparin sodium is above 2.0.The specification defined in the USP 37 and the EP 8.0 for porcine enoxaparin is not less than 1.8. Thus the sulfate to carboxylate ratio in ovine enoxaparin sodium is slightly higher, indicating a higher degree of sulfation in ovine enoxaparin sodium.

Anticoagulant activity of the ovine enoxaparin sodium is analyzed using method from USP 37. Anti-Xa activity is between 90-125 units per mg on dry basis, and anti-IIa activity is between 20-35 units per mg on dry basis, and the anti-Xa/anti-IIa ratio is between 2.8-4.8. Both the anti-Xa activity and the anti-IIa activity are within the enoxaparin sodium specifications defined in the USP 37 and the EP 8.0 for porcine enoxaparin. But the anti-Xa/anti-IIa ratio is slightly smaller, may fall outside of the specification defined in USP 37 and EP 8.0. In the USP 37 and the EP 8.0, the anti-Xa/anti-IIa ratio is between 3.3-5.3.

Preferably, the ovine enoxaparin sodium may be processed and fractionated, so that the anti-Xa activity and the anti-IIa activity as well as the ratio of both meet the enoxaparin sodium specifications defined in the USP 37.

Molecular weight and molecular weight distribution of the ovine enoxaparin sodium are analyzed using method from the USP 37. The ovine enoxaparin sodium has a weight average molecular weight of between 3800-5000, with molecular weight of <2000 being between 12.0%-20.0%, with molecular weight of >2000 and <8000 being between 68.0%-82.0%, and with molecular weight of >8000 being not more than 18.0%. The molecular weight and molecular weight distribution of the ovine enoxaparin sodium meet the enoxaparin sodium specifications defined in the USP 39 and the EP 8.6.

The 1,6-anhydro content of the ovine enoxaparin sodium is determined specifically by SAX-HPLC analysis after heparinases digestion. The digestion and analysis are performed following the “1,6-anhydro derivative inspection of enoxaparin sodium” in appendix <207> in the USP32. The 1,6-anhydro content of the ovine enoxaparin sodium is between 15%-25%, and meets the enoxaparin sodium specification defined in the USP 37 and the EP 8.0.

Preferably, the ovine enoxaparin sodium may be further processed, including decoloration, and repeated alcohol precipitation, and anion exchange fractionation or ultrafiltration fractionation, to obtain ovine enoxaparin sodium products that meet the enoxaparin sodium specifications set in the USP 37 and the EP 8.0. This will improve anti-Xa/anti-IIa ratio and other properties, so the ovine enoxaparin can completely meet those specifications in current editions of the USP 39 and the EP 8.6.

Preferably, the ovine enoxaparin sodium is for the prevention and treatment of coagulation and thrombosis-related diseases, as well as a halal anticoagulant and anti-thrombosis medicine.

A preparation method of the ovine enoxaparin sodium described above is the same as the preparation method claimed in a previous patent application (application publication No. CN 105131153 A) to the present inventors, and specifically comprises:

S1, pretreatment of raw material ovine heparin: ovine heparin sodium crude is dissolved, the solution is decolored and filtered, and then precipitated at room temperature, the precipitate is collected and dried to give ovine heparin;

S2, preparation of ovine heparin quaternary ammonium salt: the ovine heparin obtained in S1 is dissolved and formulated into an aqueous ovine heparin solution, the aqueous solution is mixed with an aqueous benzethonium chloride solution, filtration or centrifugation is performed to give ovine heparin quaternary ammonium salt, and the salt is washed and dried to give ovine heparin quaternary ammonium salt;

S3, preparation of ovine heparin benzyl ester: the dried ovine heparin quaternary ammonium salt obtained in S2 is mixed with organic solvents methylene chloride and benzyl chloride in a weight ratio for esterification, a solution of sodium acetate in methanol is dropwise added to the esterification solution of ovine heparin quaternary ammonium salt to generate ovine heparin benzyl ester precipitate, and the ovine heparin benzyl ester precipitate is filtered, washed and dried to give ovine heparin benzyl ester; and

S4, preparation of ovine enoxaparin sodium product: the ovine heparin benzyl ester in S3 is subjected to alkaline depolymerization, decoloration, neutralization with an acid, alcohol precipitation, and drying, to give ovine enoxaparin sodium product.

Preferably, in S1, ovine heparin sodium crude is dissolved using an aqueous sodium chloride solution at a weight concentration of 1%-3% for decoloration and filtration. After the decolorization, the aqueous ovine heparin sodium solution is clear and its color is not deeper than the standard color No.5; and a precipitating agent can be one of methanol, ethanol, isopropanol, or acetone, or a combination thereof. In S2, a weight ratio of benzyl chloride and ovine heparin sodium is 2-5:1. In S3, the esterification temperature is 30-40° C., and a weight ratio of ovine heparin quaternary ammonium salt, methylene chloride, and benzyl chloride is 1:3-10:1.1. In S4, the depolymerization is performed using a sodium hydroxide solution, where the depolymerization temperature is between 30° C.-70° C. and the holding time is above 0.5 h. In S4, the decoloration is performed using hydrogen peroxide, where 30% hydrogen peroxide is added in 0.1-1 times of the weight of ovine heparin benzyl ester at or below room temperature, and oxidation and decoloration last for above 10 min until the color of the reaction solution is below Y6 and GY6.

Preferably, in S3, the washing of ovine heparin benzyl ester precipitate comprises the steps of:

S31, adding methanol into the ovine heparin benzyl preparation mixture after sodium acetate-methanol treatment, standing, sedimentation, and separation, to give ovine heparin benzyl ester;

S32, adding an 8%-12% aqueous sodium chloride solution into the ovine heparin benzyl ester isolated from S31 for reconstitution, a weight ratio of the aqueous sodium chloride solution to the ovine heparin quaternary ammonium salt being 0.5-2:1;

S33, alcohol precipitation of the solution obtained in S32 with a 60%-70% final concentration of methanol; and

S34, repeating reconstitution with the aqueous sodium chloride solution and alcohol precipitation and crystallization for 2-5 times until the reconstituted ovine heparin benzyl ester is not turbid.

An ovine enoxaparin sodium injection, comprising ovine enoxaparin sodium as described above and water for injection as components.

Preferably, the ovine enoxaparin sodium injection is prepared by dissolving ovine enoxaparin sodium in Water For Injection (WFI), replenishing water for injection to a certain concentration after complete dissolution, aseptic filtration, and filling in syringes, vials or ampules.

Preferably, the ovine enoxaparin sodium injection has an activity of 10000 anti-Xa units per mL, and is preferably prepared into prefilled syringes of 4000 anti-Xa units, 6000 anti-Xa units, 10000 anti-Xa units, and other specifications.

Preferably, the ovine enoxaparin sodium injection is used as Halal anticoagulant and anti-thrombosis medicine.

Another ovine enoxaparin sodium injection comprises ovine enoxaparin sodium, water for injection and benzyl alcohol as components.

Preferably, the other ovine enoxaparin sodium injection is prepared by dissolving ovine enoxaparin sodium with water for injection, adding benzyl alcohol, replenishing water for injection to a certain concentration after complete dissolution and uniform mixing, aseptic filtration, and filling in vials.

Preferably, the concentration of benzyl alcohol is between 1.35 mg/ml and 1.65 mg/ml.

Preferably, the other ovine enoxaparin sodium injection has an activity of 10000 anti-Xa units per mL, and is preferably filled into vials of 30000 anti-Xa units and other specifications.

Preferably, the other ovine enoxaparin sodium injection is used as Halal anticoagulant and anti-thrombus medicine.

Preferably, the anticoagulant activity of the ovine enoxaparin sodium and ovine enoxaparin sodium injections is tested in vitro in human blood. After plasma is separated from blood, the effect of the ovine enoxaparin sodium and ovine enoxaparin sodium injections on blood coagulation properties, including, not limited to, APTT, TT and PT, is measured by an automatic coagulation machine using coagulation kit.

Preferably, the anticoagulant activity of the ovine enoxaparin sodium and ovine enoxaparin sodium injections is tested in vivo in animals, preferably rabbits. Preferably, after administration by subcutaneous injection, rabbit blood is collected before injection and at various points after injection, add 3.8% sodium citrate with ratio of 1:9, and loaded on the coagulation machine and tested for the effect on blood coagulation, including, not limited to, APTT, TT and PT, and other coagulation factors.

Preferably, the ovine enoxaparin sodium and ovine enoxaparin sodium injections, in both in vitro and in vivo anticoagulation tests, show similar or equivalent activity compared to the enoxaparin sodium standard.

The significance of the present invention: provided an ovine enoxaparin sodium and its injections; that meet specifications set forth for porcine enoxaparin sodium in the USP 37, and can also meet the requirements in current editions of the USP 39 and the EP 8.6 with minor chemical structures (disaccharide composition) and anticoagulant activity (smaller anti-Xa/anti-IIa ratio) difference due to its source. For ovine enoxaparin sodium, the raw material is easily available, and controllable in quality, and can significantly expand the sources and quantity of enoxaparin sodium availability for the patients. Ovine enoxaparin sodium derives from ovine, is a halal medicine, and can be used in many Muslim counties, regions and populations, with great economical potential.

A detailed description of the present invention is given below. Spectrum are included here for further description of the current invention.

### BRIEF DESCRIPTION OF THE DRAWINGS

FIG. 1 is a schematic diagram of comparison of disaccharide spectra between an ovine enoxaparin sodium sample in example 1 of the present invention and an enoxaparin sodium standard.

FIG. 2 is a schematic diagram of comparison of ¹H-NMR spectra between an ovine enoxaparin sodium sample in example 2 of the present invention and the enoxaparin sodium standard.

FIG. 3 is a schematic diagram of comparison of ¹³C-NMR spectra between an ovine enoxaparin sodium sample in example 3 of the present invention and the enoxaparin sodium standard.

FIG. 4 is a schematic diagram of sulfate to carboxylate ratio of an ovine enoxaparin sodium sample in example 4 of the present invention.

FIG. 5 is a schematic diagram of comparison of molecular weight distribution between an ovine enoxaparin sodium sample in example 6 of the present invention and the enoxaparin sodium standard.

FIG. 6 is a schematic diagram of comparison of the effects on APTT, PT and TT and anti-Xa activity in rabbits between ovine enoxaparin sodium and its injection samples in example 10 of the present invention and the enoxaparin sodium standard.

### DETAILED DESCRIPTION OF THE INVENTION

Embodiments of the present invention describe ovine-derived enoxaparin sodium—ovine enoxaparin sodium, and ovine enoxaparin sodium injections. The following experimental examples are provided to illustrate specific embodiments, and it should be understood that the specific embodiments described herein are only used to explain the present invention and are not intended to limit the present invention.

An ovine enoxaparin sodium product was derived from example 2 in the patent application (application publication No. CN 105131153 A) to present inventors. If not specified otherwise, the following examples all adopted this sample or a fractionated sample prepared by the similar process.

### EXAMPLE 1

### Analysis of Disaccharides and 1,6-Anhydro Contents in Ovine Enoxaparin Sodium:

Analysis of disaccharide composition and 1,6-anhydro content was performed following the “1,6-anhydro derivative inspection of enoxaparin sodium” in appendix <207> in the USP32. The results are shown in FIG. 1 and Table 1.

TABLE 1
Disaccharide composition and 1,6-anhydro % of ovine enoxaparin
sodium and porcine enoxaparin sodium standard

| | Percentage (%) | | | | |
|---|---|---|---|---|---|
| | 1# | 2# | 3# | 4# | 5# |
| | Re- | Re- | Re- | 1,6- | Re- |
| | duced | duced | duced | anhydro | duced |
| | ΔIVA | ΔIVS | ΔIIA | ΔIIS | ΔIIIA |
| Enoxaparin | 3.08 | 3.04 | 3.01 | 0.44 | 1.75 |
| sodium standard | | | | | |
| (STD_Porcine) | | | | | |
| Ovine | 1.52 | 3.10 | 0.79 | 0.36 | 0.58 |
| enoxaparin | | | | | |
| sodium | | | | | |
| (Ovine_038) | | | | | |

| | Percentage (%) | | | | |
|---|---|---|---|---|---|
| | 6# | 7# | 8# | 9# | 10# |
| | Re- | Re- | Re- | 1,6- | Re- |
| | duced | duced | duced | anhydro | duced |
| | ΔIIS | ΔIIIS | ΔIA | ΔIS | ΔIIA-IVS glu |
| Enoxaparin | 10.13 | 6.48 | 1.61 | 1.30 | / |
| sodium standard | | | | | |
| (STD_Porcine) | | | | | |
| Ovine | 9.19 | 5.01 | 0.91 | 1.26 | / |
| enoxaparin | | | | | |
| sodium | | | | | |
| (Ovine_038) | | | | | |

| | Percentage (%) | | | | |
|---|---|---|---|---|---|
| | 11# | | 13# | 14# | |
| | Re- | | Reduced | 1,6- | |
| | duced | 12# | ΔIIA-IIS | anhydro | 1,6- |
| | ΔIS | ΔIS | glu | ΔIS-IS | anhydro |
| Enoxaparin | 59.24 | / | 2.12 | 1.04 | 20.5% |
| sodium standard | | | | | |
| (STD_Porcine) | | | | | |
| Ovine | 70.2 | / | 1.60 | 1.11 | 20.4% |
| enoxaparin | | | | | |
| sodium | | | | | |
| (Ovine_038) | | | | | |

It can be seen from FIG. 1 and Table 1 that ovine enoxaparin sodium has the contents of disaccharides ΔIS, ΔIIS and ΔIIIS of respectively70.2%, 9.19% and 5.01%, and the content of ΔIIA-IISglu of 1.60%, which all are different from those in the enoxaparin sodium standard. Also, the 1,6-anhydro % in ovine enoxaparin sodium, 20.4%, is almost the same as that in the standard, meeting the criterion 15%-25% in the USP 37.

### EXAMPLE 2

### ¹H-NMR Analysis of Ovine Enoxaparin Sodium:

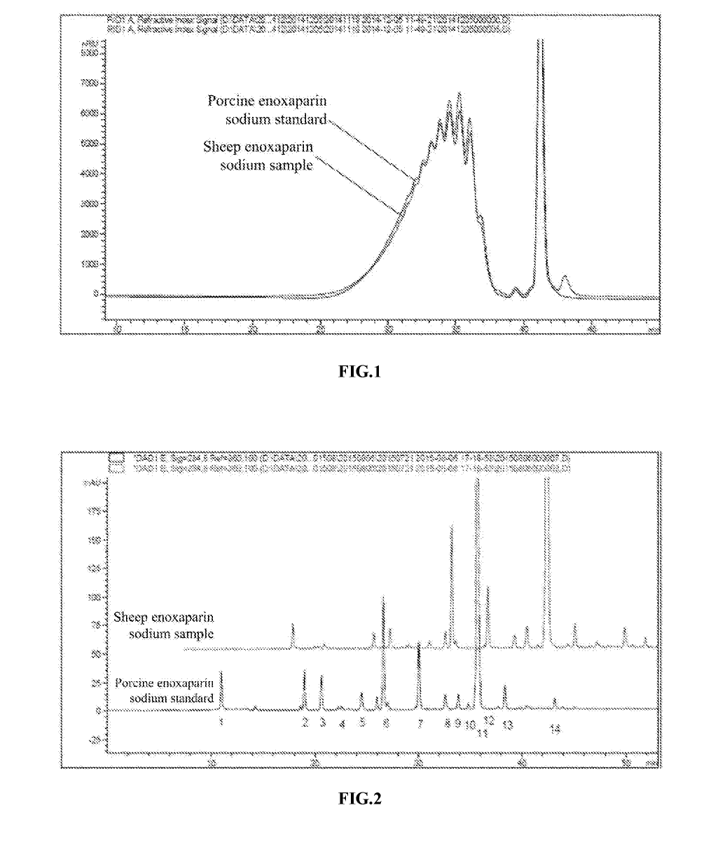

¹H-NMR analysis of ovine enoxaparin sodium was performed in a 400 MHz NMR spectrometer at the Analytical and Testing Center, Suzhou University using 3-trimethylsilyl sodium propionate -d4 (TSP) as internal reference. Formulation of a sample solution to be tested: about 20 mg of each of the ovine enoxaparin sodium sample and the enoxaparin sodium standard was accurately weighted, and dissolved with D₂O to a concentration of about 20 mg/ml by weight. 1-2 drops of TSP were dropwise added, shaken and uniformly mixed. The solution was filtered (0.22 μm) and tested. The results are shown in FIG. 2, where 3.4 ppm indicates the methyl-hydrogen peak due to residual methanol, and 4.7 ppm indicates the water-hydrogen peak.

The results show that the hydrogen spectrum of the ovine enoxaparin sodium sample is substantially identical to that of the enoxaparin sodium standard, but the integral of methyl peak in N-acetyl at δ2.04 ppm in the ovine enoxaparin sodium sample is smaller than that in the enoxaparin sodium standard, indicating that in the ovine enoxaparin sodium sample, less N-acetyl group is present, and accordingly, more N-sulfate group modification is present. Usually, more sulfate group, higher anticoagulant activity.

### EXAMPLE 3

### ¹³C-NMR Analysis of Ovine Enoxaparin Sodium:

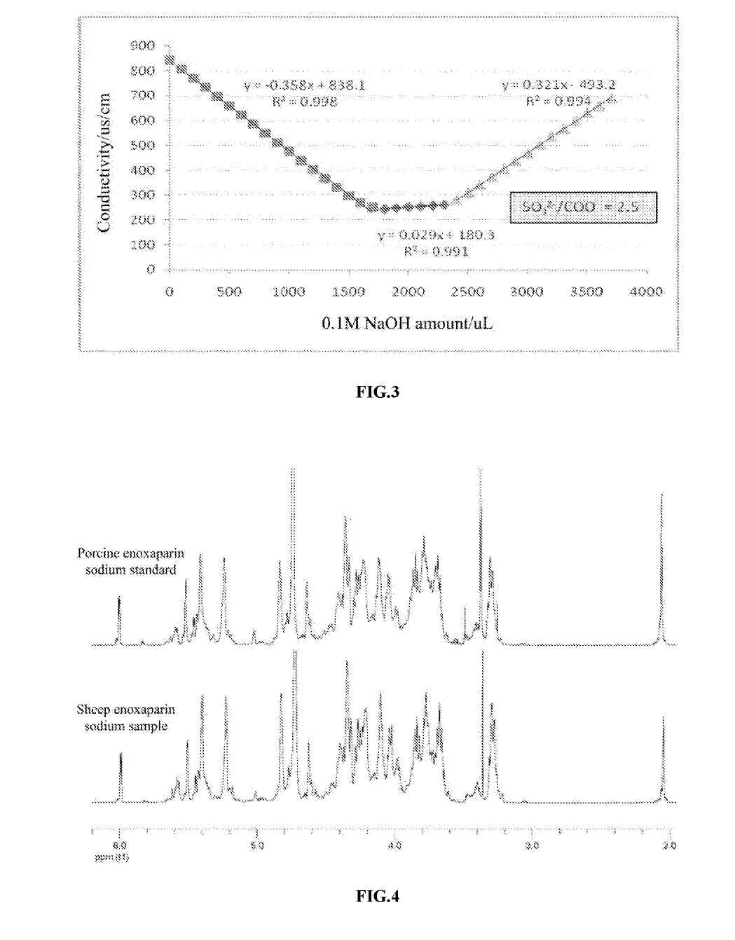

¹³C-NMR analysis of the ovine enoxaparin sodium sample was performed in a 400 MHz NMR spectrometer at the Analytical and Testing Center, Suzhou University following the method in the USP 37. The results are shown in FIG. 3, where 50 ppm indicates the methyl-carbon peak of residual methanol.

Similar to the hydrogen spectrum, the ¹³C NMR results show that carbon backbone of the ovine enoxaparin sodium sample is identical to that of the enoxaparin sodium standard, but at some specific positions, e.g., for the methyl-carbon of N-acetyl at 24.9 ppm, there is a certain difference in abundance. However the USP 37 does not have specification on integral of N-acetyl, and thus, the carbon spectrum of the ovine enoxaparin sodium sample meets the requirements in the pharmacopeias.

### EXAMPLE 4

### Analysis of Sulfate to Carboxylate Ratio of Ovine Enoxaparin Sodium:

Analysis of sulfate to carboxylate ratio was performed following the USP 37. Activated cationic resin and anionic resin were respectively packed into columns in series, with a dimension of 1.5 cm×7.5 cm and 1.5 cm×2.5 cm, respectively. 50 mg of the ovine enoxaparin sodium sample was accurately weighted and formulated with pure water to 5 mg/ml. It was loaded sequentially through the columns packed with cationic resin and anionic resin, and the eluant was collected with a beaker at the end of the columns. The eluant was titrated with a sodium hydroxide solution, and the change of conductivity was recorded. The molar ratio of sulfate to carboxylate was calculated by plotting according to the USP 37. The results are shown in FIG. 4.

It can be seen from FIG. 4 that the sulfate to carboxylate ratio of the ovine enoxaparin sodium sample is 2.4. The specification for porcine enoxaparin sodium in the USP 37 is no less than 1.8. Generally, the sulfate to carboxylate ratio of porcine enoxaparin sodium is 2.2. Thus, the sulfate to carboxylate ratio of ovine enoxaparin sodium is slightly larger, indicating more sulfation modification in ovine enoxaparin sodium.

### EXAMPLE 5

### Comparative Analysis of Chromogenic Anti-Xa Activity, Anti-IIa Activity, and Whole Ovine Plasma Activity:

Anti-Xa activity and anti-IIa activity were determined following the USP37 method, and anticoagulant activity in the whole ovine plasma was determined using the method in the patent application (application publication No. CN 105131153 A). Comparison of respective activities of the ovine enoxaparin sodium sample and the enoxaparin sodium standard is shown in table 2 below.

TABLE 2
Comparison of anticoagulant activity for ovine enoxaparin sodium sample

| | Anticoagulant | | | |
|---|---|---|---|---|
| | activity in whole | | | |
| | ovine plasma method | Anti-Xa | Anti-IIa | |
| Name | (unit per mg) | (unit per mg) | (unit per mg) | Anti-Xa/anti-IIa |
| USP37 | / | 90.0-125.0 | 20.0-35.0 | 3.3-5.3 |
| specification | | | | |
| Standard | 43.0 | 100.6 | 29.2 | 3.4 |
| (enoxaparin sodium, | | | | |
| STD_Porcine) | | | | |
| Ovine enoxaparin | 45.9 | 105.6 | 29.0 | 3.6 |
| sodium (Ovine-038) | | | | |

The results show that the anticoagulant activity in the whole ovine plasma method of the ovine enoxaparin sodium sample is comparable to that of the enoxaparin sodium standard, and the ovine enoxaparin sodium sample meets the USP 37 specifications for anti-Xa activity, anti-IIa activity and anti-Xa/anti-IIa ratio provided in the pharmacopeias.

### EXAMPLE 6

### Analysis of Weight Average Molecular Weight and Molecular Weight Distribution:

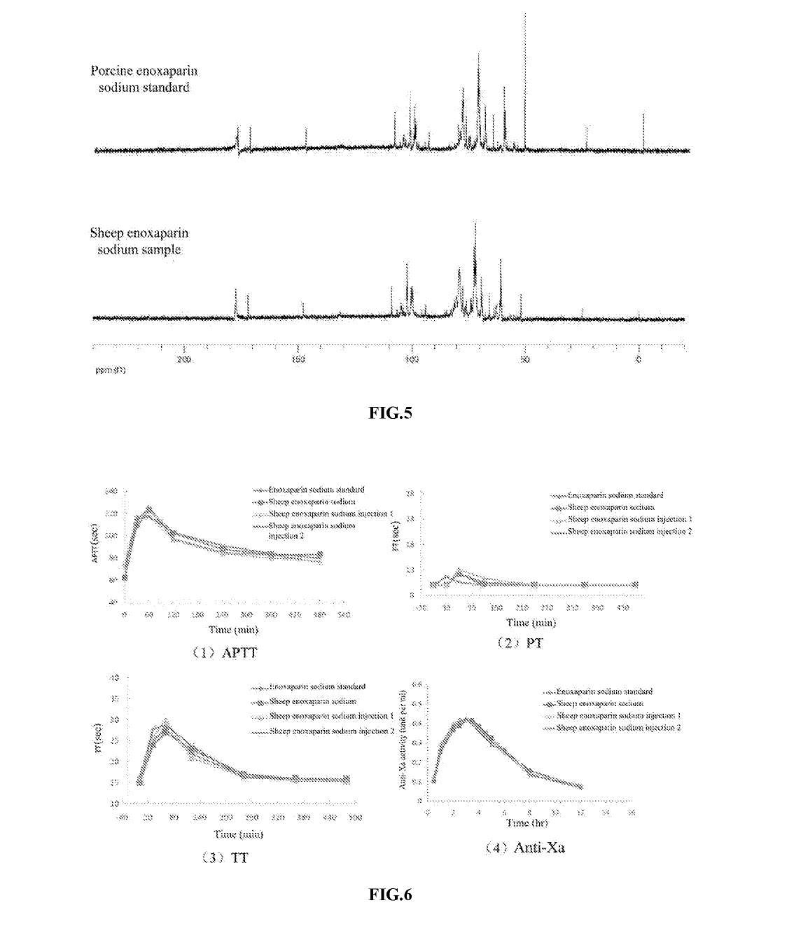

Analysis and calculation of molecular weight and molecular weight distribution were performed using the method in the USP 37. The results are shown in FIG. 5 and Table 3.

TABLE 3
Weight average molecular weight and molecular weight
distribution of ovine enoxaparin sodium sample

| | Weight average | | | | |
|---|---|---|---|---|---|
| | molecular | | <2000 | 2000-8000 | >8000 |
| Item | weight (Da) | | Da | Da | Da |
| USP37 specification | 3800-5000 | Da | 12.0-20.0% | 68.0-82.0% | ≤18.0% |
| Standard (porcine | 4299 | Da | 17.4% | 71.2% | 10.4% |
| intestinal mucosa | | | | | |
| enoxaparin sodium, | | | | | |
| STD_Porcine) | | | | | |
| Ovine enoxaparin | 4237 | Da | 17.3% | 73.0% | 9.7% |
| sodium (Ovine-038) | | | | | |

The results show that the weight average molecular weight and the molecular weight distribution of the ovine enoxaparin sodium sample (Ovine-038) are very close to those of the enoxaparin sodium standard, are within specifications in the USP 37.

### EXAMPLE 7

### Preparation of Ovine Enoxaparin Sodium Injection 1:

190.6 g (loss on drying of 3.4%, 105.6 anti-Xa units per mg on dry basis, 20 million anti-Xa units in total) of ovine enoxaparin sodium powder was calculated for activity and accurately weighted, dissolved with cold water for injection and made up to 2000 ml, aseptically filled through two-stage 0.2 micron filters into a level A clean zone, and filled into 1 ml glass syringes by a filling machine with a specification of 6000 units (or 0.6 ml). 2160 products of the ovine enoxaparin sodium injection 1 were obtained in total (excluding the loss).

### EXAMPLE 8

### Preparation of Ovine Enoxaparin Sodium Injection 2:

190.6 g (loss on drying of 3.4%, 105.6 anti-Xa units per mg on dry basis, 20 million anti-Xa units in total) of ovine enoxaparin sodium powder was calculated for activity and accurately weighted, dissolved with cold water for injection, added with 75.0 g benzyl alcohol, uniformly stirred, and then made up to 2000 ml with cold water for injection, aseptically filled through two-stage 0.2 micron filters into a level A clean zone, and filled into 5 ml vials by a filling machine with a specification of 30000 units (or 3.0 ml). 552 products of the ovine enoxaparin sodium injection 2 were obtained in total (excluding the loss).

### EXAMPLE 9

### In Vitro Anticoagulation Tests Using Human Blood Forovine Enoxaparin Sodium and Ovine Enoxaparin Sodium Injections:

Experimental method: 3 parts of 3 mL peripheral venous blood were collected each time, added 3.8% sodium citrate (with ratio of 1:9), and centrifuged for 5 min at 3000 rpm to separate platelet poor plasma (PPP). According to the instruction from the kit provider, it was loaded to an automatic coagulation machine(Stago Compact) and tested. Experimental groups are as follows: ovine enoxaparin sodium sample group (lot number: Ovine-038), ovine enoxaparin sodium injection 1 group (described in example 7), ovine enoxaparin sodium injection 2 group (described in example 8), and enoxaparin sodium standard group (a commercial clinical drug, Clexane, lot number: 24459), all with a concentration of ˜3 μg/mL. In the experiments, saline was used as blank control.

Results and Analysis:

1) APTT, PT and TT

The results are shown in a table below:

TABLE 4
Effect on APTT, PT and TT in vitro

| Group | APTT | | PT | TT | |
|---|---|---|---|---|---|
| Ovine enoxaparin | 104.1 ± 9.5 | s | 13.2 ± 0.6 s | 127.4 ± 37.4 | s |
| sodium sample | | | | | |
| Ovine enoxaparin | 105.3 ± 12.1 | s | 13.9 ± 0.4 s | 145.4 ± 43.6 | s |
| sodium injection 1 | | | | | |
| Ovine enoxaparin | 102.5 ± 9.8 | s | 13.4 ± 0.5 s | 134.4 ± 44.3 | s |
| sodium injection 2 | | | | | |
| Enoxaparin sodium | 104.8 ± 10.2 | s | 13.8 ± 0.3 s | 140.4 ± 54.7 | s |
| Blank control | 38.1 ± 1.4 | s | 12.7 ± 0.6 s | 16.6 ± 0.7 | s |

As shown in table 4 that all samples significantly prolong APTT and TT, but have a little effect on PT.

2) AT and Fibrinogen:

The results are shown in the table below:

TABLE 5
Effect on AT and fibrinogen in vitro

| Group | AT | Fibrinogen | | Recalcification time | |
|---|---|---|---|---|---|
| Ovine enoxaparin | 2.31 ± 0.34 g/L | 96.33 ± 10.50 | s | 31.00 ± 0.00 | s* |
| sodium sample | | | | | |
| Ovine enoxaparin | 2.26 ± 0.34 g/L | 97.00 ± 9.54 | s | 31.00 ± 0.00 | s* |
| sodium injection 1 | | | | | |
| Ovine enoxaparin | 2.24 ± 0.37 g/L | 96.67 ± 9.50 | s | 31.00 ± 0.00 | s* |
| sodium injection 2 | | | | | |
| Enoxaparin sodium | 2.29 ± 0.44 g/L | 99.00 ± 6.29 | s | 31.00 ± 0.00 | s* |
| Blank control | 2.57 ± 0.25 g/L | 94.00 ± 8.1 | s | 9.95 ± 0.40 | s |

*out of detection limit

As shown in table 5 that compared to the enoxaparin sodium standard, ovine enoxaparin sodium and its injections have almost no effect on AT and fibrinogen; however all samples can significantly prolong the recalcification time, and all data are out of detection limit (>31.00 s).

All data above reveal that ovine enoxaparin sodium and its injections have significant anticoagulant effect in vitro, and are comparable to the enoxaparin sodium standard.

### EXAMPLE 10

### In Vivo Anticoagulation Tests in Animals Forovine Enoxaparin Sodium and Ovine Enoxaparin Sodium Injections:

Experimental method: 2-3 Kg Japanese White Rabbits were chosen, and respectively administered by subcutaneous injection at antedorsal near upper limbs based on body weight. Experimental groups are as follows: ovine enoxaparin sodium sample group (lot number: Ovine-038), ovine enoxaparin sodium injection 1 group (described in example 7), ovine enoxaparin sodium injection 2 group (described in example 8), and enoxaparin sodium standard group (a commercial clinical drug, Clexane, lot number: 24459), all with a concentration of 1 mg/Kg. In the experiments, saline was used as blank control. 3 mL blood was collected before-injection and 30 min, 1 h, 2 h, 4 h, 6 h, and 8 h post-injection respectively, added 3.8% sodium citrate (with ration of 1:9), loaded and tested (identical to Section 1.1).

Experimental Results and Analysis:

1) APTT:

The experimental results are shown in FIG. 6(1). It can be seen from the figure that compared to the enoxaparin sodium standard, ovine enoxaparin sodium and its injections all can significantly prolong APTT, and are comparable in prolonging APTT, and have a similar time at which APTT reaches the maximum and a similar decay time in rabbits, revealing that ovine enoxaparin sodium and its injections are comparable with the enoxaparin sodium standard in rabbits.

2) PT:

The experimental results are shown in FIG. 6(2). It can be seen from the figure that all groups of samples have no effect on PT in rabbits. In addition, in Section 1.2 above, these three samples did not significantly prolong PT in vitro. That is consistent with in vivo result.

3) TT:

The experimental results are shown in FIG. 6(3). It can be seen from the figure that ovine enoxaparin sodium and its injections all can significantly prolong TT, and compared to the enoxaparin sodium standard, have a similar time at which TT reaches the maximum and a similar decay time in rabbits.

4) Anti-Xa Activity:

The experimental results are shown in FIG. 6(4). It can be seen from the figure that anti-Xa activity of heparin in rabbit plasma, and absorption and metabolism (decay) profiles for all samples are comparable after subcutaneous injection, all reach absorption peak values at about 2 h to 4 h and nearly eliminated at 8 h.

All data above demonstrated that ovine enoxaparin sodium and its injections have comparable in vivo anticoagulant effects as the enoxaparin sodium standard.

The present invention may be implemented in many embodiments, and any technical solution obtained by equivalent substitution or modification shall fall within the protection scope of the present invention.

What is claimed is:

1. An ovine enoxaparin sodium, wherein the ovine enoxaparin sodium is prepared from ovine heparin.

2. (canceled)

3. The ovine enoxaparin sodium of claim 1, wherein an integral of methyl peak of N-acetyl at δ2.04 ppm in ¹H-NMR spectrum and δ24.9 ppm in ¹³C-NMR spectrum of the ovine enoxaparin sodium is smaller than that of a porcine enoxaparin sodium.

4. The ovine enoxaparin sodium of claim 1, wherein a ratio of sulfate to carboxylate in the ovine enoxaparin sodium is greater than 2.0.

5. The ovine enoxaparin sodium of claim 1, wherein the ovine enoxaparin sodium has an anti-Xa activity of 90-125 units per mg on dry basis, and an anti-IIa activity of 20-35 units per mg on dry basis, and a ratio of anti-Xa/anti-IIa is 3.3-5.3.

6. The ovine enoxaparin sodium of claim 1, wherein the ovine enoxaparin sodium has an average molecular weight of 3800-5000, wherein 12.0%-20.0% wt of the ovine enoxaparin sodium has a molecular weight of less than 2000, 68.0%-82.0% wt of the ovine enoxaparin sodium has a molecular weight of greater than 2000 and less than 8000, no more than 18% wt of the ovine enoxaparin sodium has a molecular weight of greater than 8000; and a content of a 1,6-anhydro is 15%-25%.

7. The ovine enoxaparin sodium of claim 1, comprising 66%-74% wt of disaccharide ΔUA2S-GlcNS6S (ΔIS), 8%-10% wt of disaccharide ΔUA-GlcNS6S (ΔIIS), and 4%-6% wt of disaccharide ΔUA2S-GlcNS (ΔIIIS).

8. The ovine enoxaparin sodium of claim 7, comprising 66.24% wt of disaccharide ΔUA2S-GlcNS6S (ΔIS), 9.15% wt of disaccharide ΔUA-GlcNS6S (ΔIIS), and 6.44% wt of disaccharide ΔUA2S-GlcNS (ΔIIIS).

9. A method for preparing an ovine enoxaparin sodium, comprising the steps of:
S1, pretreating: dissolving an ovine heparin sodium crude to obtain a solution, decolorizing the solution, filtering, then alcohol precipitating at room temperature, collecting a precipitate thereof, drying to obtain the ovine heparin;
S2, preparing an ovine heparin quaternary ammonium salt: dissolving the ovine heparin obtained in S1 to obtain an aqueous solution, mixing the aqueous solution with an aqueous benzethonium chloride solution, filtering, washing and drying to obtain the ovine heparin quaternary ammonium salt;
S3, preparing an ovine heparin benzyl ester: mixing the ovine heparin quaternary ammonium salt obtained in S2 with an organic solvent consisting of methylene chloride and benzyl chloride in a predetermined weight ratio for esterification to obtain an esterification mixture, dropwise adding a solution of sodium acetate methanol solution to the esterification mixture to generate an ovine heparin benzyl ester precipitate, filtering the ovine heparin benzyl ester precipitate, washing and drying to obtain the ovine heparin benzyl ester; and
S4, preparing the ovine enoxaparin sodium: subjecting the ovine heparin benzyl ester obtained in S3 to an alkaline depolymerization, decoloring, neutralizing with an acid, alcohol precipitating, and drying to obtain the ovine enoxaparin sodium.

10. The method of claim 9, wherein in S1, the ovine heparin sodium crude is dissolved using an aqueous sodium chloride solution with a weight concentration of 1%-3% for decolorization, filtration, until an aqueous solution of the ovine heparin sodium is clear and a color thereof is not deeper than the standard color No.5.

11. The method of claim 9, wherein in S1, a precipitating agent for the alcohol precipitating is one or more selected from the group consisting of methanol, ethanol, isopropanol, and acetone.

12. The method of claim 9, wherein in S2, a weight ratio of the benzyl chloride to ovine heparin sodium is 2-5:1.

13. The method of claim 9, wherein in S3, a temperature of the esterification is 30-40° C., and a weight ratio of the ovine heparin quaternary ammonium salt, methylene chloride, to benzyl chloride is 1:3-10:1.1.

14. The method of claim 9, wherein in S3, the washing comprises the sub-steps of:
S31, adding methanol into the esterification mixture after dropwise adding the sodium acetate methanol solution, standing for sedimentation and separation, to obtain the ovine heparin benzyl ester;
S32, adding an aqueous sodium chloride solution with a concentration of 8%-12% w/v into the ovine heparin benzyl ester isolated in S31 for reconstitution, a weight ratio of the sodium chloride solution to the ovine heparin quaternary ammonium salt is 0.5-2:1;
S33, alcohol precipitating and crystallizing the solution obtained in S32 with a 60%-70% vol methanol; and
S34, repeating the sub-steps S32 and S33 for 2-5 times until a reconstitution solution of the ovine heparin benzyl ester is not turbid.

15. The method of claim 9, wherein in S4, the depolymerization is performed using a sodium hydroxide solution at a depolymerization temperature of 30° C.-70° C. for more than 0.5 h.

16. The method of claim 9, wherein in S4, the decolorization is performed using hydrogen peroxide by adding 10%-100% wt of 30% hydrogen peroxide into the ovine heparin benzyl ester at or below room temperature to obtain a reaction solution for oxidation and decolorization for more than 10 minutes, until the color of the reaction solution is below Y6 and GY6.

17. (canceled)

18. An ovine enoxaparin sodium injection, comprising an ovine enoxaparin sodium of claim 1 and water for injection.

19. (canceled)

20. The ovine enoxaparin sodium injection of claim 18, further comprising benzyl alcohol.

21. (canceled)
